(19) United States
(12) Plant Patent Application Publication
(10) Pub. No.: US 2010/0306891 P1
(43) Pub. Date: Dec. 2, 2010

(54) Calluna plant named 'SALSA'

(50) ;

(76) Inventor: Christian Robert Nielsen, Soltau (DE)

Correspondence Address: PLANT HAVEN, INC., P.O. BOX 3056, SANTA BARBARA, CA, 93130, US

(21) Appl. No.: 12/804,659

(22) Filed: Jul. 27, 2010

Related U.S. Application Data
(60) Provisional application No. 61/273,281, filed on Aug. 3, 2009.

(30) Foreign Application Priority Data
Sep. 5, 2008 (DE) CLL390

(51) Int. Cl. A01H 5/00

(52) U.S. Cl. PLT/231

(57) ABSTRACT

A new cultivar of CALLUNA named ‘SALSA’ that is distinguishable by compact habit, dark green foliage, and dark red double flowers that remain in bud throughout their season. In combination these traits set ‘SALSA’ apart from all other existing varieties of Calluna known to the inventor.

This application claims the benefit of priority under 35 U.S.C. 119(e) of U.S. Provisional Application No. 61/273,281 filed on Aug. 3, 2009, entitled Calluna Plant Named ‘SALSA’.

### GENUS

CALLUNA

### SPECIES

vulgaris

### DENOMINATION

‘SALSA’

### BACKGROUND OF THE INVENTION

The present invention relates to a new and distinct cultivar of bud flowering CALLUNA, an ornamental known commonly as scots heather that is grown for garden and landscape. The new invention from the heather family Ericaceae, is known botanically as CALLUNA vulgaris and will be referred to hereinafter by the cultivar name ‘SALSA’.

‘SALSA’ was discovered by the inventor in 2005 as a naturally occurring vegetative sport within a growing crop of the inventor's previous selection of CALLUNA vulgaris ‘Flamenco’. Discovery took place in a cultivated area of Soltau, Germany. The parent is an individual Calluna vulgaris ‘Flamenco’ (unpatented). ‘SALSA’ is distinguishable from the parent by darker flower bud color and darker foliage color.

‘SALSA’, was selected by the inventor based on the criteria of long lasting, double flower buds, dark red flower bud color, and compact growth habit. The two closest comparison plants are Calluna ‘Dark Star’ (unpatented) and Calluna ‘Dark Beauty’ (unpatented). ‘SALSA’ is distinguishable from ‘Dark Beauty’ by compact habit, double flower buds, and absence of single flowered sports. ‘SALSA’ is distinguishable from ‘Dark Star’ by double flower buds, dark flower bud color, and long lasting flower buds.

‘SALSA’ exhibits compact growth habit, dark green foliage, long lasting dark red double flowers that remain unopened buds throughout their season. Leaves are small and scale-like tightly adpressed to the stems, and flower buds crowded on raceme. At one year ‘SALSA’ reaches 8 cm. in height and within the range of 10-12 cm. in width. At maturity ‘SALSA’ is 20 cm. in height and within the range of 30-40 cm. in width. ‘SALSA’ performs best in ericaceous growing media, and is hardy in USDA Zone 4.

‘SALSA’ was first asexually propagated in 2005 in a cultivated area of Soltau, Germany. Asexual propagation was accomplished by the inventor using the method of internodal semi-hardwood cuttings. Since that time under careful observation, the distinguishing characteristics of ‘SALSA’ have been determined stable, uniform, and have shown to reproduce true to type in successive generations of asexual propagation.

### SUMMARY OF THE INVENTION

The following traits have been repeatedly observed and represent the distinguishing characteristics of ‘SALSA’. These traits in combination distinguish ‘SALSA’ from all other existing varieties of CALLUNA known to the inventor. ‘SALSA’ has not been tested under all possible conditions and phenotypic differences may be observed with variations in environmental, climatic, and cultural conditions, however, without any variance in genotype.

1. Plants of ‘SALSA’ exhibit a compact growth habit.

2. The foliage of ‘SALSA’ is dark green in color.

3. Plants of ‘SALSA’ bear dark red flower buds.

4. The flower buds of ‘SALSA’ are double and long lasting.

5. At one year plants of ‘SALSA’ are 8 cm. in height and 10-12 cm. in width.

6. At maturity plants of ‘SALSA’ are 20 cm. in height and 30-40 cm. in width.

7. ‘SALSA’ performs best in ericaceous growing media.

### BRIEF DESCRIPTION OF THE DRAWINGS

The accompanying color drawings illustrate the overall appearance of ‘SALSA’ showing color as true as is reasonably possible to obtain in color reproductions of this type. Color in the drawings may differ from the color values cited in the detailed botanical description, which accurately describe the actual color of ‘SALSA’. Drawings are made from 13 to 14-month-old plants grown from rooted cuttings out-of doors.

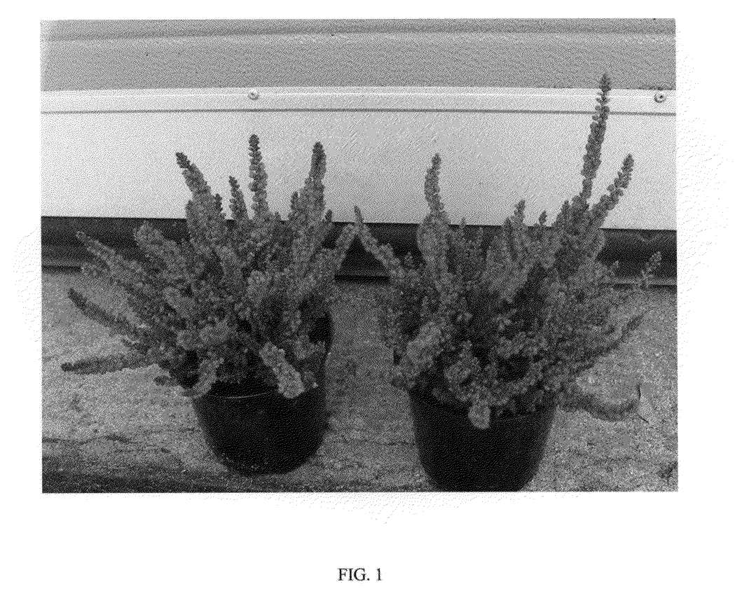

Drawing labeled FIG. 1 depicts two blooming container plants in Soltau, Germany.

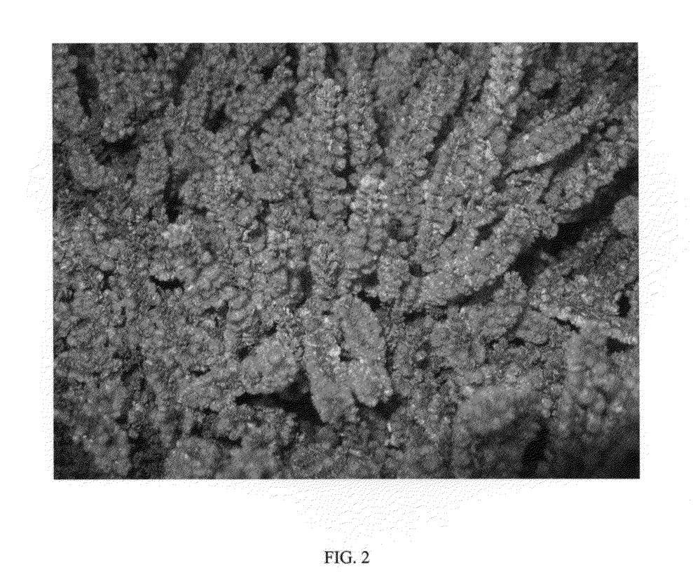

Drawing labeled FIG. 2 depicts a close-up view of the flowers. Drawings were made using conventional techniques and although flower and foliage color may appear different from actual color due to light reflectance, they are as accurate as possible by conventional photography.

### BOTANICAL DESCRIPTION OF THE PLANT

The following is a detailed botanical description of ‘SALSA’. Observations, measurements, values and comparisons were collected in Arroyo Grande, Calif. in July 2010 from 10 cm. container plants growing out-of-doors in a shaded area. Color determinations are made in accordance with The 2001 Royal Horticultural Society Colour Chart from London, England, except where general color terms of ordinary dictionary significance are used.
Botanical classification: CALLUNA vulgaris ‘SALSA’.
Family: Ericaceae.
Genus: CALLUNA.
Species: vulgaris.
Denomination: ‘SALSA’.
Common name: Scots heather.
Habit: Compact.
Plant type: Perennial.
Commercial category: Ornamental.
Use: For garden and landscape.
Suggested commercial container size: 10 cm. and 1-litre containers.
Parentage: CALLUNA vulgaris ‘SALSA’ was discovered as a naturally occurring vegetative sport resulting from the parent, an individual Calluna vulgaris ‘Flamenco’ (unpatented).
Propagation method: Internodal semi-hardwood cuttings.
Rooting system: Fine and fibrous.
Vigor: Vigorous.
Time to develop roots: 14-20 days are needed for an initial cutting to develop roots.
Temperature to develop roots: The recommended air temperature is 20-21° Centigrade.
Crop time (range): 10 weeks to 2 months are needed to produce a 10-centimeter container from a rooted cutting.
Plant dimensions (first year): 8 cm in height and 10-12 cm. in width.
Plant dimensions (at maturity): 20 cm. in height and 30-40 cm. in width. Cultural requirements: Performs best in ericaceous growing media under shade with moderate water.
Pest or disease susceptibility: Susceptible to the fungal disease Phytophthora.
Hardiness: USDA Zone 4.
Stem:
   Branching.—Basal.
   Young stem color.—176A.
   Mature stem color.—N199B.
   Stem length (average).—15 cm.
   Stem diameter.—3 mm.
   Stem shape.—Cylindrical.
   Young stem surface.—Pubescent.
   Mature stem surface.—Scurfy.
   Internode length (average).—3 mm.
Foliage:
   Type.—Evergreen.
   Leaf arrangement.—Opposite.
   Leaf division.—Simple.
   Margin.—Entire.
   Leaf shape.—Scale-like and tightly adpressed to stems.
   Leaf length.—3 mm.
   Leaf width.—1 mm.
   Leaf base.—Truncate.
   Leaf apex.—Acute.
   Venation.—Inconspicuous.
   Leaf surface (abaxial).—Puberulent.
   Leaf surface (adaxial).—Puberulent.
   Leaf attachment.—Sessile.
   Leaf color (both surfaces).—Ranges between and including 138A and N138B.
   Stipules.—Absent.
   Fragrance.—None observed.
Inflorescence:
   Inflorescence type.—Bracteate raceme.
   Inflorescence quantity.—5 per 10 cm. container plant.
   Inflorescence length (average).—6 cm.
   Inflorescence width (average).—0.60 cm.
   Blooming seasons.—Late summer through winter.
   Peduncle length (average).—6 cm.
   Peduncle width.—1 mm.
   Peduncle shape.—Cylindrical.
   Peduncle surface.—Lanate.
   Peduncle color.—183A.
   Peduncle strength.—Strong, flexible.
   Corolla color.—N74A.
   Corolla aspect.—Facing outward.
   Corolla shape (range).—Globular to urceolate.
   Corolla surface.—Pubescent.
   Corolla dimensions.—1.50 mm. in diameter.
   Corolla quantity (average on 10 cm. container plant).—20 per inflorescence.
   Corolla depth.—2 mm.
   Corolla diameter.—1.80 mm.
   Petal quantity.—5 in number.
   Petal shape.—Oblong.
   Petals fused or unfused.—Unfused.
   Petal apex (range).—Retuse to rounded.
   Petal margin.—Entire.
   Petal dimensions.—1 mm. in width and 2 mm. in length.
   Petal color (abaxial and adaxial surfaces).—N74A.
   Petal surface (abaxial and adaxial).—Glabrous.
   Calyx.—5 sepals.
   Sepal apex.—Acute.
   Sepal base.—Truncate.
   Bracteole.—Bilobular.
   Bracteole length.—2 mm.
   Bracteole color.—138A.
   Bracteole surface (abaxial and adaxial).—Pubescent.
   Fragrance of inflorescence.—None observed.
Reproductive organs (covered by corolla):
   Stamens (average).—10 in number.
   Stamen color.—155C.
   Ovary position.—Superior.
Seed: None observed.

1. A new and distinct cultivar of Calluna plant named ‘SALSA’ as described and illustrated herein.
